(19) United States
(12) Patent Application Publication
(10) Pub. No.: US 2026/0114913 A1
(43) Pub. Date: Apr. 30, 2026

(54) MAGNET ACTUATED CRANIAL (M.A.C.) DISTRACTION SYSTEM

(71) Applicant: Cornell University, Ithaca, NY (US)

(72) Inventor: Mohammed A. Fouda, Ithaca, NY (US)

(21) Appl. No.: 19/368,766

(22) Filed: Oct. 24, 2025

Related U.S. Application Data
(60) Provisional application No. 63/711,473, filed on Oct. 24, 2024.

(51) Int. Cl. A61B 17/88; A61B 17/00

(52) U.S. Cl. CPC A61B 17/885; A61B 2017/00128; A61B 2017/00398; A61B 2017/00477; A61B 2017/00876

(57) ABSTRACT

According to the present disclosure, a medical distraction system is provided. The system can include a distractor embedded entirely under the skin of a patient and a driver located entirely outside the skin of a patient. The driver can be configured to drive motion of the distractor via magnetic coupling with the distractor.

### CROSS REFERENCE TO RELATED APPLICATIONS

This application claims priority to and the benefit of U.S. Provisional Patent Application No. 63/711,473, filed 24 Oct. 2024, the disclosure of which is now expressly incorporated herein by reference.

### FIELD OF THE DISCLOSURE

The present disclosure relates generally to medical devices, and more specifically to bone distraction systems.

### BACKGROUND

Distraction osteogenesis is a process used to repair skeletal deformities. The process includes slowly separating bone and allowing new bone to form in the gap between the separated bone segments. In some cases, distraction osteogenesis using traditional systems involves risks, such as infection and system failure. There is a need for improved systems for use in distraction osteogenesis.

### SUMMARY

The present disclosure may comprise one or more of the following features and combinations thereof.

According to the present disclosure, a medical distraction system is provided. The system can include a distractor and a driver configured to drive motion of the distractor via magnetic coupling with the distractor.

The distractor can be adapted to be fixed to bone segments and placed in its entirety under the skin of a patient. The distractor can be configured to displace one bone segment relative to another. The distractor can include a fixed base configured to be fixed to a first bone segment, a mobile base configured to be fixed to a second bone segment and having a rod that extends to the fixed base along a rod axis, and a gear train configured to drive motion of the mobile base relative to the fixed base via interaction with the rod. The gear train can be configured to drive motion of the mobile base away from the fixed base via interaction with the rod.

In illustrative embodiments, the gear train of the distractor can include an input magnet mounted for rotation about an input axis that is different from the rod axis. The gear train can include a worm gear fixed to the input magnet for rotation therewith. The gear train can include a drive nut having external gear teeth and internal threads engaged with the rod. The rod can be formed to include threads engaged with the internal threads of the drive nut.

In illustrative embodiments, the gear train can include a transfer gear assembly. The transfer gear assembly can include a worm wheel engaged with the worm gear and a transfer gear having gear teeth engaged with the external gear teeth of the drive nut.

In illustrative embodiments, the fixed base can include a base plate with holes therethrough sized to receive fasteners and a rod guide defining a passage along the rod axis sized to receive the rod therethrough. The rod guide can include a first frame having a first aperture defining a portion of the passage and a second frame having a second aperture defining another portion of the passage, the second aperture being spaced apart from the first aperture along the passage. The gear train can be arranged to engage the rod along the rod axis between the first aperture and the second aperture. The gear train can be coupled to the fixed base.

In illustrative embodiments, the driver can be adapted to remain in its entirety external to the patient. The driver can include a housing, a knob coupled to the housing for rotation about a driver axis, and a drive magnet coupled to the knob for rotation about the driver axis and configured to magnetically couple to the input magnet of the distractor while the distractor is under the skin of the patient and the driver remains external to the skin of the patient.

In illustrative embodiments, the driver can include a transmission coupled between the knob and the driver magnet. The transmission can have a gearbox configured to convert a single rotation of the knob into more than one rotation of the drive magnet.

In illustrative embodiments, the transmission can include a slip clutch. The slip clutch can be configured to selectively decouple the drive magnet from the knob when torque developed between the drive magnet and the knob exceed a preselected threshold.

In illustrative embodiments, the driver can include a feedback unit configured to provide auditory and/or haptic feedback indicative of the amount of knob rotation relative to the housing so that a user can monitor expected rotation of the drive magnet along with the associated motion of the distractor during operation of the system. The feedback unit can include (i) a detent fixed to one of the knob or the housing and (ii) an elastic protrusion fixed to the other of the knob or the housing, the elastic protrusion arranged to be contacted by the detent upon rotation of the knob relative to the housing. The elastic protrusion can be provided by a spring-loaded ball.

In illustrative embodiments, the driver can include a magnet positioner unit configured to selectively change the distance from the driver magnet to an end of the housing. The magnet positioner unit can be made up of a cap that forms the end of the housing, the cap being mounted to a housing body to move relative to the housing body along the driver axis. The cap can be coupled to the housing body via cam slots shaped to move the cap along the driver axis in response to rotation of the cap about the driver axis.

A distractor adapted to be fixed to bone segments and placed in its entirety under the skin of a patient is a further part of the present disclosure. The distractor can be configured to displace one bone segment relative to another.

In illustrative embodiments, the distractor can include a fixed base configured to be fixed to a first bone segment, a mobile base configured to be fixed to a second bone segment and having a rod that extends to the fixed base along a rod axis, and a gear train configured to drive motion of the mobile base relative to the fixed base via interaction with the rod. The gear train can have an input magnet configured to drive motion of the mobile base away from the fixed base via interaction with the rod.

In illustrative embodiments, the input magnet is mounted for rotation about an input axis that is different from the input axis. The input axis can be spaced apart from the rod axis. The input axis can be perpendicular to the rod axis.

In illustrative embodiments, the gear train can include a worm gear fixed to the input magnet for rotation therewith and a drive nut having external gear teeth and internal threads engaged with external threads formed on the rod. The gear train can include a transfer gear assembly including a worm wheel engaged with the worm gear and a transfer gear having gear teeth engaged with the external gear teeth of the drive nut.

In illustrative embodiments the fixed base can include a rod guide defining a passage along the rod axis sized to receive the rod therethrough. The rod guide can include a first frame having a first aperture defining a portion of the passage and a second frame having a second aperture defining another portion of the passage, the second aperture being spaced apart from the first aperture along the passage. The gear train is arranged to engage the rod along the rod axis between the first aperture and the second aperture. The gear train can include a drive nut having external gear teeth and internal threads engaged with external threads formed on the rod, the drive nut being arranged around the rod axis between the first aperture and the second aperture.

These and other features of the present disclosure will become more apparent from the following description of the illustrative embodiments.

### BRIEF DESCRIPTION OF THE DRAWINGS

FIG. 1 is a perspective view of a magnet actuated cranial distraction system applied to an illustrative skull showing that the system includes a distractor adapted to be fixed to cranial bone segments under a patient's skin and a driver adapted to remain external to the patient while being able to drive motion of the distractor via magnetic coupling with the distractor;

FIG. 2 is an enlarged view of the magnet actuated cranial distraction system showing that the distractor includes a fixed base, a mobile base with a threaded rod, and a gear train with an input magnet configured to drive motion of the mobile base away from the fixed base via interaction with the threaded rod; and showing that the driver includes a drive magnet in a housing and a drive knob;

FIG. 3 is an exploded perspective assembly view of the distractor from FIGS. 1 and 2 showing that the distractor includes the fixed base with a rod guide adapted to allow the threaded rod to slide therethrough, the mobile base with the threaded rod fixed thereto, and the gear train; and showing that the input magnet is part of an input assembly, a transfer assembly, and an output assembly;

FIG. 4 is a detailed perspective assembly view of the fixed base showing that the fixed base includes a base plate with screw holes, the rod guide made up of a left frame integrated with the base plate and a right frame fixed to the left frame via fasteners, and a number of bushings to support rods/shafts for rotation relative to the fixed base;

FIG. 5 is a detailed perspective assembly view of the input assembly of the gear train showing that the input assembly includes the input magnet fixed to a magnet platform by a pin, a worm gear, and an input shaft that couples the input magnet to the worm gear;

FIG. 6 is a detailed perspective assembly view of the transfer assembly of the gear train showing that the transfer assembly includes a worm wheel adapted to be driven by the worm gear of the input assembly, a transfer (spur) gear, and a transfer shaft that couples the worm wheel to the transfer gear for rotation together;

FIG. 7 is a detailed perspective assembly view of the output assembly included in the gear train showing that the output assembly includes a drive nut with external gear teeth adapted to be driven by the transfer gear and internal threads adapted to engage with external threads of the threaded rod and thereby drive motion of the mobile base;

FIG. 8 is a perspective view of the driver with portions broken away to show that the driver includes a transmission interconnecting the drive knob at the top of the drive housing to the drive magnet arranged at the bottom;

FIG. 9 is a perspective assembly view of the driver showing that the transmission interconnecting the knob and the drive magnet includes a gearbox configured to convert a single rotation of the knob to multiple rotations of the drive magnet and a slip clutch configured to decouple the knob from the drive magnet at a driver torque threshold preselected to be lower than a maximum torque able to be carried from the drive magnet in the driver to the input magnet in the distractor during use of the system;

FIG. 10 is perspective view of the magnet actuated cranial distraction system applied to an illustrative skull showing that, in operation, the knob of the driver can be rotated in a first direction to cause distraction (movement apart) of adjacent bones in a patient's skull and in a second direction to cause compression (movement together) of adjacent bones in a patient's skull.

### DETAILED DESCRIPTION OF THE DRAWINGS

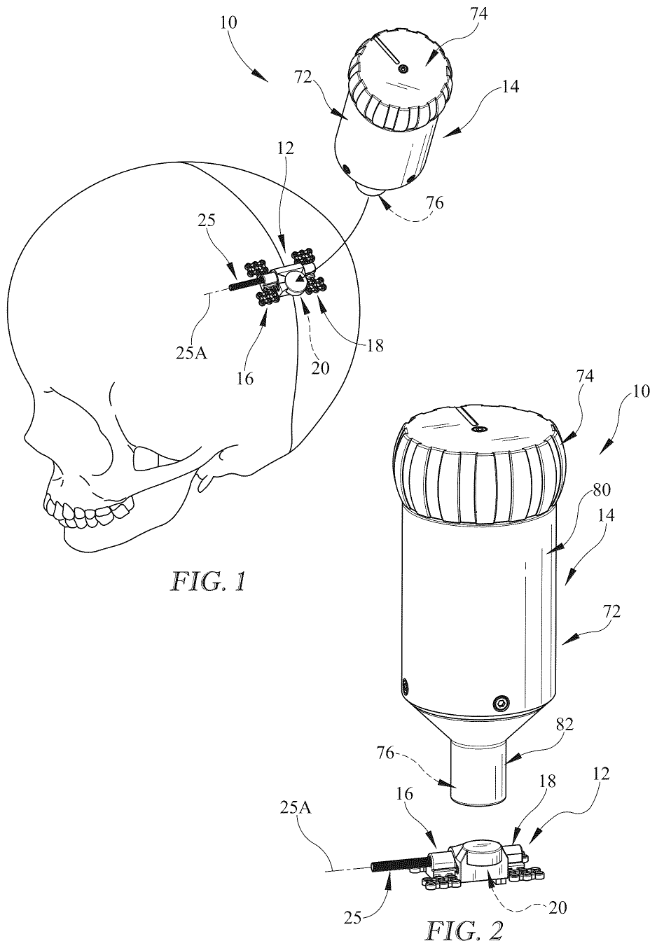

In an illustrative embodiment, a magnet actuated cranial distraction system 10 is shown in FIGS. 1, 2, and 10. The system 10 includes a distractor 12 for implantation entirely under the skin of a patient and a driver 14 for use with the distractor 12 while remaining entirely external to the patient. The distractor 12 is configured to displace one bone segment relative to another. The driver 14 is configured to drive motion of the distractor 12 via magnetic coupling through the skin of the patient with the distractor 12.

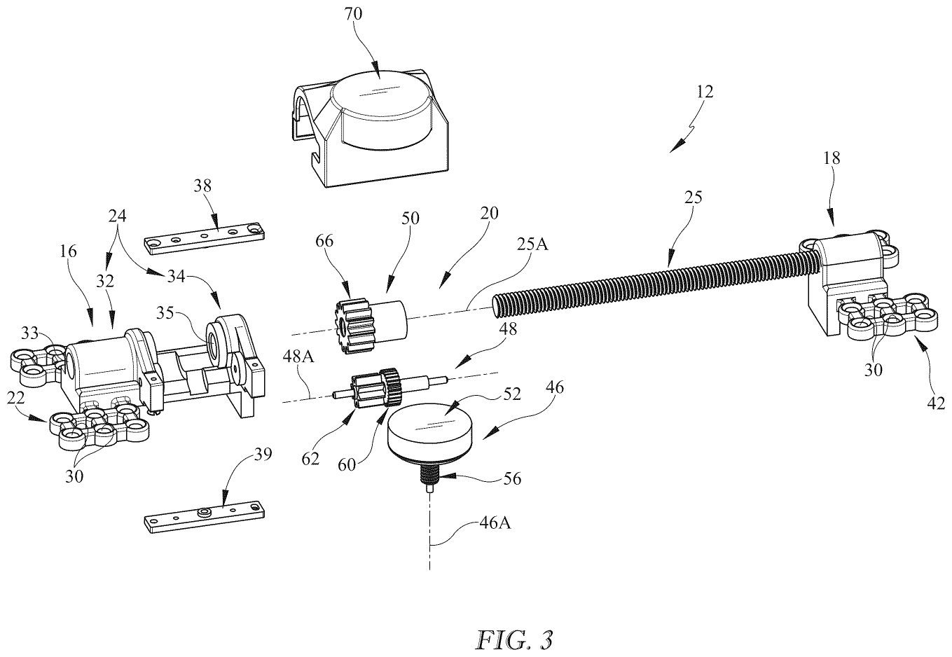

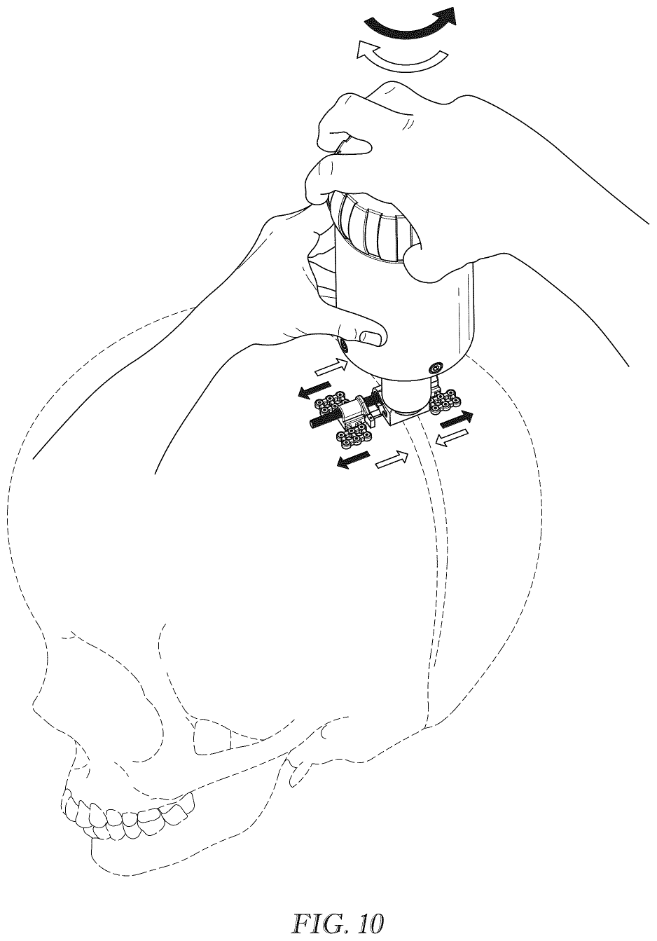

The distractor 12 is operable for distraction (movement apart) or compression (movement together) of adjacent bones as suggested in FIG. 10. The distractor 12 illustratively includes a fixed base 16, a mobile base 18, and a gear train 20 as shown in FIG. 3. The fixed base 16 is adapted to be coupled to a first bone segment via fasteners. The mobile base 18 is adapted to be fixed to a second bone segment and has a threaded rod 25 that extends to the fixed base 16 along a rod axis 25A. The gear train 20 is configured to drive motion of the mobile base 18 relative to the fixed base 16 via interaction with the threaded rod 25.

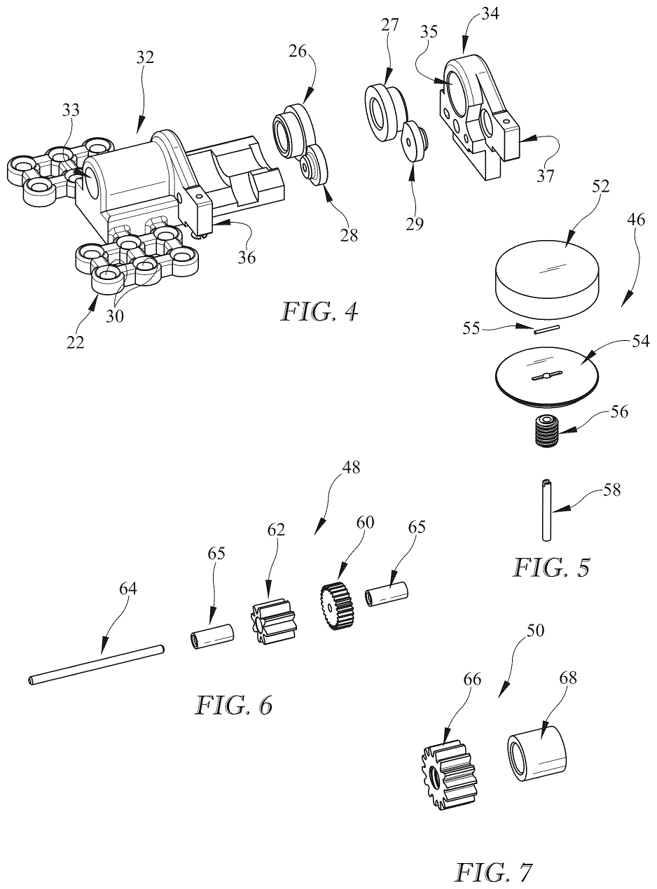

The fixed base 16 includes a base plate 22, a rod guide 24, and bushings 26, 27, 28, 29 as shown in FIG. 4. The base plate 22 is formed to include holes 30 configured to receive self tapping screws for fixing the base plate 22 to a bone. The rod guide 24 receives the threaded rod 25 therethrough and the bushings 26, 27 facilitate sliding of the threaded rod 25 along the rod axis 25A.

The rod guide 24 includes a left frame 32 having a first aperture 33 and a right frame 34 having a second aperture 35. The left frame 32 is spaced apart from the right frame 34 along the rod axis 25A and the gear train 20 interacts with the threaded rod 25 in the spaced defined therebetween. The apertures 33, 35 cooperate to define a passageway through the rod guide 24 aligned with the rod axis 25A.

In the illustrative embodiment, the fixed base 16 also includes protrusions 36, 37 and plates 38, 39 that support rotating pieces of the gear train 20 as suggested in FIGS. 2 and 3. The protrusions 36, 37 are formed as part of frames 32, 34 and support a rotor parallel to the rod axis 25. The protrusions 36, 37 have bushings 28, 29 therein to minimize friction. The top and bottom plates 38, 39 are fixed to the protrusions 36, 37 and support a rotor of the gear train 20 perpendicular to the rod axis 25.

The fixed base 16 is a precision-milled aluminum structure incorporating an array of clearance apertures 30 for bone screw fixation, thereby securing the contralateral end of the distractor 12 to the patient. Unlike the mobile base 18, the drive rod 25 traverses an unthreaded bore 33, 35 lined with polyoxymethylene (POM) bushings, which permit low-friction axial translation. Two lateral protrusions 32, 34 house precision-machined bores 33, 35 that constrain the gearbox shafts 58, 64, while a distal protrusion 32—located furthest from the mobile base 18—serves as a thrust-bearing surface for the translating nut 66 responsible for generating distraction forces. Additional recesses and relief cuts accommodate the motion envelope of the gearbox 20 components without impeding their operation.

The fixed base 16 (sometimes called a left frame) employs a two-piece architecture to facilitate gearbox 20 assembly. The distal segment incorporates the majority of structural features and interfaces, whereas the proximal segment functions primarily as a mounting plate. These two halves are joined via a precision pin-and-slot registration interface and secured with a single M1.6 screw. Once assembled, they form a monolithic structural unit that is not intended to be disassembled. Like the mobile base (sometimes called the right frame), the left frame contains no moving parts, serving purely as a rigid, load-bearing support for the mechanical elements of the distraction assembly 12.

The mobile base 18 is configured to be pushed away from or pulled toward the fixed base 16 during operation of the distractor 12 as shown in FIG. 10. The mobile base 18 includes a base plate 42 and the threaded rod 25 as shown in FIG. 3. The base plate 42 is formed to include holes 30 configured to receive self tapping screws for fixing the base plate 22 to a bone. The threaded rod 25 is fixed to the base plate and extends along the rod axis 25A. It is contemplated that the rod 25 could have indentations, teeth, or even a smooth outer surface. In any case, the rod 25 is configured to be engaged by the gear train 20 via threads, gear teeth, rollers, or other outputs to drive motion of the mobile base 18.

In the exemplary implementation of the disclosed design, the mobile base 18 is a precision-milled aluminum baseplate with an array of clearance holes 30 for fixation using self-tapping screws, and a 316 stainless steel M3.5 drive rod 25 secured within a threaded bore. Although devoid of moving parts, it plays a critical structural role by providing a stable, load-bearing anchor point for the distraction mechanism, thereby ensuring accurate force transmission and positional stability throughout actuation

The gear train 20 is configured to be magnetically coupled to the driver 14 and to drive motion of the mobile base 18 included in the distractor 12 as suggested in FIG. 10. The gear train 20 illustratively incudes an input assembly 46, a transfer assembly 48, and an output assembly 50 as shown in FIGS. 3 and 5-7. The input assembly 46 magnetically couples to the driver 14 and rotates on an input axis 46A. The transfer assembly 48 is engaged with the input assembly 46 and the output assembly 50 to step up/down rotation speed as well as to change rotational direction of the gear train 20. The output assembly 50 directly engages the threaded rod 25 to drive motion relative to the rod guide 24 of the fixed base 16.

The input assembly 46 is mounted to plates 38, 39 for rotation about an input axis 46A that is spaced apart from and perpendicular to the rod axis 25A. The input assembly 46 illustratively includes input magnet 52, a manet platform 54 coupled to the internal magnet by a pin 55, a worm gear 56, and an input shaft 58 as shown in FIG. 5. The input magnet 52 is configured to magnetically couple to the driver 14 during use of the system 10. It is contemplated that the input magnet 52 could be omitted and a ferrous (or otherwise magnetically active) magnet platform 54 could be magnetically coupled to the driver 14 during use of the system 10. The worm gear 56 is fixed to the input magnet 52 for rotation therewith along the input axis 46A established by the shaft 58.

The transfer assembly 48 illustratively modifies the rotational speed and changes the rotational direction received by the input assembly 46 moving to the output assembly 50 as suggested in FIG. 3. The transfer assembly 48 includes a worm wheel 60, a transfer (spur) gear 62, a transfer shaft 64, and transfer spacers 65 as shown in FIG. 6. The worm wheel 60 engages and is driven by the worm gear 56 of the input assembly 46. The transfer gear 62 turns with the worm wheel 60 and drives the output assembly 50. The transfer shaft 64 supports the transfer assembly 48 via the protrusions 36, 37 for rotation about a transfer axis 48A that is parallel to and spaced apart from the rod axis 25A.

The output assembly 50 is driven by the transfer assembly 48 and rotates about the rod axis 25A as suggested in FIG. 3. The output assembly includes a drive nut 66 and a spacer 68 as shown in FIG. 7. The drive nut 66 has internal threads engaged with external threads of the rod 25 and external gear teeth engaged with the transfer gear 62. In the illustrative embodiment, the drive nut 66 is trapped between the left and right frames 32, 34 of the rod guide 24 so that force developed by interaction of the drive nut 66 threads and the rod 25 threads are reacted through the fixed base 16 in into a patient's bone. In this way, upon rotation of the drive nut 66, the rod 25 and mobile base 18 are pushed or pulled along the axis 25A.

The gearbox 20 houses all of the distractor's active (rotating) mechanical components and is responsible for converting magnetic input into controlled linear displacement. At its uppermost interface is a diametrically magnetized N42-grade neodymium magnet 52 (12.7 mm diameter×3.2 mm thick). The magnet is bonded to a precision-machined aluminum platform 54 using cyanoacrylate adhesive, which is subsequently secured to the stainless-steel input shaft 58 with a wicking anaerobic thread locker. A stainless-steel keying pin 55 mechanically couples the components, ensuring reliable torque transmission.

A steel single-start worm gear 56 (module=0.2 mm, pitch diameter=2.4 mm, length=5.5 mm) is bonded to the input shaft 58 with thread locker and constitutes the first stage of torque reduction (FIG. 14). The input shaft assembly 46 is captured between two steel mounting plates 38, 39 affixed to the left frame 16 by M1.6 screws. One end of each plate is slotted, allowing fine adjustment of the worm-wheel mesh clearance. The worm 56 engages a brass worm wheel 60 (module=0.2 mm, 26 teeth, face width=2 mm), which is bonded to a secondary stainless-steel shaft 64 to form a compound gear assembly with an adjacent spur gear 62.

The spur gear 62, fabricated from brass (module=0.5 mm, 8 teeth, face width=4 mm), is axially positioned by a polyoxymethylene (POM) spacer 65, and POM bushings supporting the shaft 64 to minimize friction and wear. This spur gear 62 drives a final 14—tooth brass gear 66 (module=0.5 mm, face width=4 mm) whose internal bore is threaded (M3.5) so that it functions as a translating nut 66 (FIG. 16). Rotation of this gear 66 directly advances the distraction drive rod 25. The final gear is axially constrained by a POM spacer 68 on the proximal side and bears against a POM bushing 26 on the distal side of the left frame 16, where the distraction force is applied.

The overall gear reduction ratio of the gear train 20 is 45.5:1, and, given the drive rod's 25 thread pitch of 0.6 mm, the assembly produces a linear advancement of approximately 0.013 mm per revolution of the input shaft 58. This high reduction enables precise, incremental distraction under controlled loading conditions, a critical requirement for safe and predictable cranial bone transport.

The exemplary distractor 12 also includes a cover (or housing) 70 as shown in FIG. 3. The cover 70 is coupled to the fixed base 16 and extends over the gear train 20 between the left and right frames 32, 34 where the drive nut 66 engages the threaded rod 25. The cover 70 protects the gear train 20 from liquid and/or ingrowth of a patient onto moving parts of the distractor 12.

A custom-fabricated cover 70, produced via high-resolution 3D printing in a MicroFine ABS-like resin, encloses the left frame 16 and its associated gear train 20. Its primary function is to shield the internal components from particulate contamination and mechanical damage, while also providing a barrier against fluid ingress. This protective enclosure enhances the durability and reliability of the device in the operative environment without compromising its mechanical performance

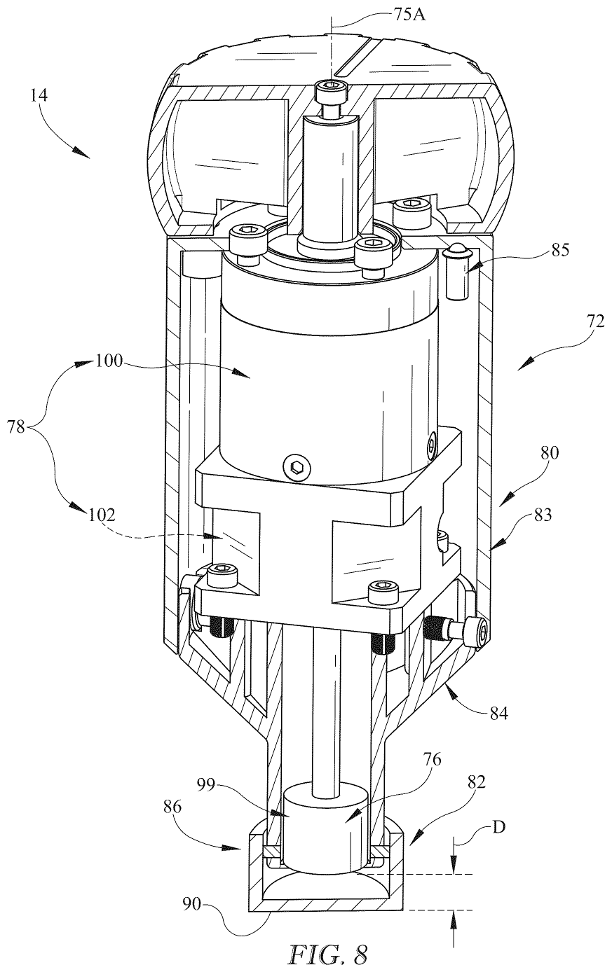

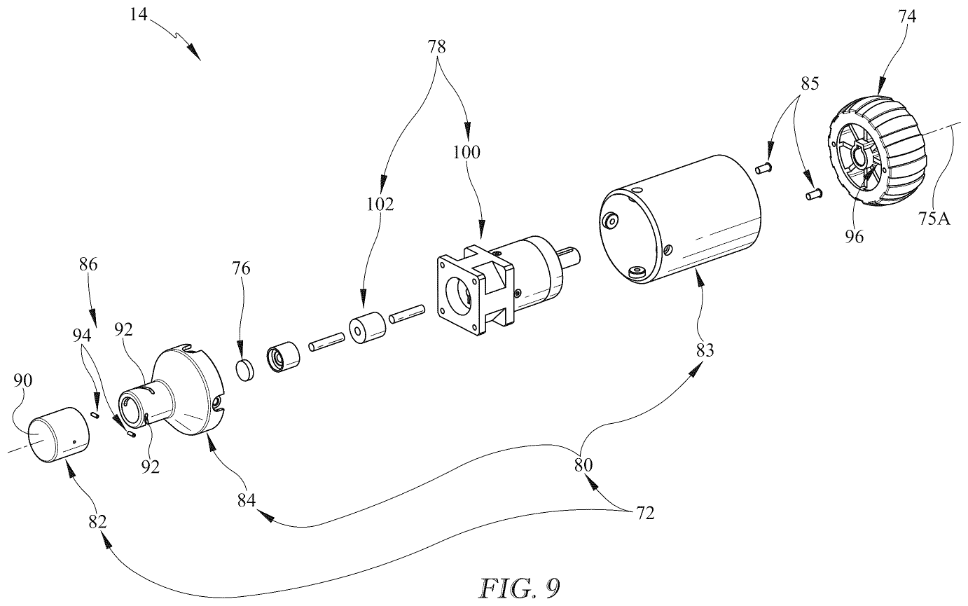

Turning now to the driver 14 of the system 10, it will be appreciated that various different devices can be used with the distractor 12 while remaining within the scope of the present disclosure. The driver 14 illustratively includes a housing 72, a knob 74, a driver magnet 76, and a transmission 78 as shown in FIGS. 8 and 9. The knob 74 and driver magnet 76 are mounted to the housing 72 for rotation relative to the housing 72 about a driver axis 75A. The transmission 78 couples the knob 74 to the driver magnet 76 and controls relative motion of the driver magnet 76 and the knob 74.

The housing 72 is illustratively made up of a housing body 80 and a cap 82 as shown in FIG. 9. The housing body 80 includes a body piece 83 and a neck piece 84. The body piece 83 has two spring-loaded ball plungers 85 arranged to interface with the knob 74. The spring-loaded ball plungers 85 provide an elastic protrusion that can interact with the knob 74. The cap 82 is movable relative to the housing body 80 about and along the driver axis 75A so as to provide an optional magnet positioner unit 86.

The magnet positioner unit 86 is configured to selectively change the distance D from the driver magnet 76 to an end 90 of the housing 72 as suggested in FIGS. 8 and 9. The magnet positioner unit 86 also includes cam slots 92 formed in the neck piece 84 of the housing body 80 and pins/fasteners 94 that extend from the cap 82 into the cam slots 92 as suggested in FIG. 9. The cam slots 92 are shaped to move the cap 82 along the driver axis 75A in response to rotation of the cap 82 about the driver axis 75A.

The knob 74 in the exemplary embodiment is mounted to the body piece 83 of the housing body 80 for rotation about the driver axis 75 as shown in FIGS. 8 and 9. The knob 74 includes a grip 95 and a detent 96 that extends from the grip 95 toward the housing body 80. The detent 96 and the spring-loaded ball plungers 85 cooperate to provide an optional feedback unit 98 configured to provide auditory and/or haptic feedback indicative of the amount of knob 74 rotation relative to the housing 72. The spring-loaded ball plungers 85 are arranged to be contacted by the detent 96 upon rotation of the knob 74. In this way, a user can monitor expected rotation of the drive magnet 76 along with the associated motion of the distractor 12 during operation of the system 10.

The driver magnet 76 is configured to magnetically couple to the input magnet 52 of the drive train 20 while the distractor 12 is under the skin of the patient and the driver 14 remains external to the skin of the patient. In the illustrative embodiment, the driver magnet 76 is received in a cup 99 fixed to the neck piece 84 of the housing body 80 as shown in FIG. 8. The driver magnet 76 is free to rotate about the driver axis 75A within the cup 99.

The transmission 78 is coupled between the knob 74 and the driver magnet 76 as shown in FIG. 8. The transmission 78 includes a gearbox 100 and a slip clutch 102 as shown in FIGS. 8 and 9. The gearbox 100 is illustratively configured to convert a single rotation of the knob 74 into more than one rotation of the drive magnet 76. The slip clutch 102 is configured to selectively decouple the drive magnet 76 from the knob 74 when torque developed between the drive magnet 76 and the knob 74 exceed a preselected threshold.

All externally visible components of the driver 14 are fabricated from a high-resolution, ABS-like photopolymer via additive manufacturing. The housing 72 consists of two primary body segments 83, 84, while a rotating control knob 74 forms the third and final exterior element. At the core of the driver 14 is a single-stage planetary gearbox 100 with an 8:1 reduction ratio, which serves as the fundamental torque-transmission element.

Torque input is delivered through a 5 mm stainless steel shaft that couples directly to a machined stainless-steel cup 99, within which a 12.7 mm diameter×3.2 mm thick, N42-grade diametrically magnetized neodymium magnet 76 is bonded using cyanoacrylate adhesive. For overload protection, a slip clutch mechanism 102 is integrated between the gearbox 100 output and the magnet interface. The gearbox 100 input flange is fastened to the lower housing segment 84 with four M3 screws, while the upper housing 83 slides into place from above and is similarly secured with four M3 screws. The external control knob 74 attaches directly to the gearbox 100 output shaft via an M3 screw.

Because the knob 74 is mounted at the gearbox output, the driver 14 functions as a speed multiplier rather than a reducer, and its single-stage, low-reduction configuration permits easy back drivability. Two spring-loaded ball plungers 85 embedded within the main housing 83 engage detent features 96 on the knob 74, producing two tactile clicks per revolution to provide haptic feedback during operation.

An optional positioning feature 86 can be integrated at the distal tip 90 of the driver 14: a removable plastic cap 82 mounted via a quarter-turn, bayonet-style axial cam 92, 94. This cap 82 enables controlled, incremental positioning of the driver magnet 76 relative to the internal distractor 12, thereby reducing the risk of sudden magnetic engagement. The cam tracks 92 are machined directly into the lower housing 84, and the cap 82 itself is retained by two M2 set screws 94, which simultaneously function as cam pins.

Theoretical Model: The present disclosure allows for determination of the dimensions of the device to ensure the generation of the required output force (F) for a given input torque (T). In the MAC, the input torque must overcome two primary sources of mechanical energy dissipation before producing an axial distraction force: (1) thread friction along the helical interface and (2) collar (thrust-bearing) friction at the rotating thrust interface. Therefore, the total torque demand is generated as Tₜₒₜₐₗ=T_{thread}+T_{collar}.

Thread Geometry: Thread geometry and torque requirements influence the efficiency and mechanical integrity of the device. The relationship between thread characteristics—such as diameter, pitch, and material properties—and the required torque is well-established in engineering mechanics, particularly in lead screw and force transmission-based distraction systems.

Thread Diameter: A smaller thread diameter (dₘ) reduces frictional forces by decreasing the contact area between the screw and nut. This reduction in contact area minimizes the friction-induced energy loss, lowering the input torque required from the external magnet. However, reducing the thread diameter also increases mechanical stress, leading to possible premature wear and deformation. The mechanical stress in the threads can be approximated using the following equation:

$$σ = \frac{F}{{A}_{t}} ,$$

where σ=Axial stress in the thread (Pa) and At=Thread engagement area (m²)—depending on the thread profile and depth.

Thread Pitch and Lead Angle: A larger thread pitch (l) increases the lead angle of the screw (λ), which in turn affects the torque transmission efficiency. Lead angle influence—

$$λ = {tan}^{- 1} ( \frac{l}{π {d}_{m}} )$$

—represents the lead angle of the screw, which affects the efficiency of force transmission. However, suppose the lead angle exceeds the self-locking condition given by tan (λ)<f. In that case, the system will no longer be self-locking, potentially leading to unintended reversals in motion due to external forces. Therefore, the pitch must be carefully optimized to balance ease of actuation with stability.

### Torque Requirements ∥

Thread Friction—The axial force transmitted at the distractor-bone interface is determined to be F=74N. The mean thread diameter (dₘ) of the driving screw is 3.25 mm with a lead (l): 0.6 mm, and a coefficient of friction (f): 0.35. The torque required to overcome thread friction, drive the nut, and advance the driving screw can be calculated using the following formula from Design of Machine Elements by M.F. Spotts:

$${T}_{thread} = \frac{F {d}_{m}}{2} \frac{l + π {fd}_{m}}{π {d}_{m} - f l}$$

$${T}_{thread} = \frac{74 \cdot 3.25 \times {10}^{- 3}}{2} \frac{6.0 \times 1 {0}^{- 4} + π ( 0.35 ) ( 3.25 \times 1 {0}^{- 3} )}{π ( 3.25 \times 1 {0}^{- 3} ) - ( 0.35 ) ( 6.0 \times 1 {0}^{- 4} )} = 50.2 mN m$$

Collar Friction—In compact gear-driven distraction systems, collar torque can consume 15-30% of the total available torque, substantially altering the relationship between applied torque and axial load. This is particularly important for systems like the MAC that employ torque-limited magnetic drivers, where the coupling strength and slip-clutch threshold limit the maximum transmissible torque. Collar torque is generated as the axial load (F) produces a normal pressure field over the annular contact region between the inner radius (rᵢ) and the outer radius (r₀) of the washer (bearing face of the housing of the driving screw). The local frictional shear stress at radius (r) is τ=μ_{c}p(r), where p(r) is the local pressure and μ_{c} is the coefficient of friction at the collar interface. The incremental friction force acting on an infinitesimal ring of circumference 2πTr and thickness (dr) is dF=τ·2πr dr, which produces a differential torque dT_{collar}=r dF=2πμ_{c}p(r)r²dr. Frictional torque is generated by the tangential friction force acting on the entire annulus:

$${T}_{c o l l a r} = {\int }_{{r}_{i}}^{{r}_{o}} {μ}_{c} p ( r ) 2 π {r}^{2} dr .$$

Assuming uniform pressure—a good approximation when the washer face is stiff relative to the screw head and deflects minimally—the pressure is

$$p ( r ) = F / π ( {r}_{o}^{2} - {r}_{i}^{2} ) ⇒ {T}_{c} = \frac{2}{3} F {μ}_{c} \frac{{r}_{o}^{3} - {r}_{i}^{3}}{{r}_{o}^{2} - {r}_{i}^{2}} .$$

The effective moment arm for frictional torque generation

$$( \frac{{r}_{o}^{3} - {r}_{i}^{3}}{{r}_{o}^{2} - {r}_{i}^{2}} ) ,$$

known as the effective friction radius (r_{eff}), so that T_{collar}=Fμ_{c}r_{eff}. The effective friction diameter is then D_{c}=2r_{eff}, yielding

$${T}_{c o l l a r} = \frac{F {μ}_{c} {D}_{c}}{2} .$$

For the MAC geometry, with rᵢ=1.5 mm and r₀=3.5 mm,

$${r}_{eff} = \frac{2}{3} \frac{{3.5}^{3} - {1.5}^{3}}{{3.5}^{2} - {1.5}^{2}} = 1.99 mm$$

and D_{c}=2r_{eff}=3.98 mm. For an axial load F=74N and μ_{c}=0.10, the collar torque is:

$${T}_{c o l l a r} = \frac{( 74 ) ( 0.10 ) ( 3.98 \times 1 {0}^{- 3} )}{2} = 0.0 \; 147 N \cdot m = 14.7 mN \cdot m .$$

Therefore, the total torque is:

Tₜₒₜₐₗ=T_{thread}+T_{collar}=>Tₜₒₜₐₗ=0.0502+0.0147=0.0649N·m=64.9N·m

Mechanical Efficiency-Maintaining positional stability of a cranial distraction system under physiological loading is a fundamental requirement for safe and predictable distraction and bone regeneration. In screw-driven distraction systems, this stability is governed by the concept of self-locking, which ensures that the driving screw cannot back-drive in response to the axial loads generated by soft tissues, muscular tension, or other environmental factors.

Mechanically, a driving screw is self-locking when the lead angle (λ) is smaller than the friction angle (φ), guaranteeing that the resisting frictional moment exceeds the moment generated by the axial force, producing a net positive torque barrier that prevents reverse motion. This means that after each incremental distraction, the device remains in place, and the distracted segment is held in position. In the MAC, the lead angle (λ) is computed from

$$tan λ = \frac{l}{π {d}_{m}} ,$$

yielding

$$λ = arctan ( \frac{l}{π {d}_{m}} ) = 3.36 ° ,$$

whereas the friction angle (φ) is obtained from tan φ=f⇒φ=arctan (f)=19.29°. Since the lead angle remains well below the friction angle (λ<φ), the system satisfies the self-locking feature, ensuring that the screw cannot back-drive under physiological loading. The self-locking feature is critical for maintaining distraction stability and preventing unintended loss of distraction during the consolidation phase.

The useful energy per revolution (Fl) is the axial force times the linear advance per revolution, while the input work (2 πT) is the torque times the angular rotation per revolution. The efficiency (η) of a driving screw is defined as the ratio of useful energy output per revolution to the input energy per revolution:

$$η = \frac{useful energy / rev}{input energy / rev} ,$$

determining what fraction of input energy is converted into translational motion:

$$η = \frac{Fl}{2 π {T}_{thread}} ⟹ η = \frac{74 \cdot 6. \times {10}^{- 4}}{2 π ( 0.0502 )} = 0.141 ( 14.1 \% )$$

Subsequently, the mechanical efficiency of the system can be presented as:

$$η = \frac{Fl}{2 π {T}_{total}} = \frac{74 \cdot 6. \times {10}^{- 4}}{2 π \cdot 0.0649} = 0.109 ( 10.9 \% ) ,$$

meaning that nearly 89% of the input energy is dissipated in friction.

The critical efficiency (η_{crit}) is the maximum efficiency at which the driving screw remains self-locking. It corresponds precisely to the transition point where the lead angle (λ) equals the friction angle (φ). At this point, the torque generated by the axial load is just sufficient to overcome thread friction, meaning the screw is at the threshold of back-driving. Any efficiency above this value will result in losing the self-locking ability. For the MAC, critical efficiency is computed as

$${η}_{crit} = \frac{tan ϕ}{tan ( 2 ϕ )} ⟹ {η}_{crit} = \frac{tan ( 19.29 ° )}{tan ( 38.58 ° )} \approx 0.314 ( 31.4 \% )$$

The efficiency (η) is not merely a descriptor of energy conversion but a comprehensive indicator of device usability, mechanical safety, and clinical reliability. Beyond its role as a performance metric, efficiency provides a quantitative measure of frictional energy dissipation—which, rather than being a design drawback, serves as an intrinsic safeguard against unintended reverse motion. Explicitly reporting η and comparing it to η_{crit}, therefore, transforms device validation from a qualitative assertion of “self-locking” into a reproducible, quantitative argument.

An efficiency (η) of 10.9% means that only about 1/9 of the rotational input energy is converted into linear distraction energy, while the remaining 89% is dissipated as friction in the threads. At first glance, this may seem low, but in fact, low efficiency is a desirable feature of the MAC.

This analysis provides a quantitative design framework for selecting thread geometry and surface friction to ensure robust mechanical stability in vivo. Operating well below η_{crit} is advantageous, as it prevents back-driving. Our device, with a measured efficiency (η) of 10.9% lies way below the η_{crit} (31.4%), corresponding to a quantitative safety margin of

$$1 - \frac{η}{{η}_{c r i t}} ⟹ 1 - \frac{0.109}{0.314} = 65 \% .$$

Such a wide safety margin against unintentional reversal is advantageous, as it accommodates potential variability in friction due to lubrication by bodily fluids like seroma and blood, wear-induced friction reduction, or tolerance stack-up, which could slightly elevate efficiency during the consolidation phase.

This conservative design choice has direct clinical implications. First, it safeguards callus stability, prevents inadvertent loss of distraction, and obviates the need for secondary locking mechanisms, simplifying the device architecture and reducing manufacturing and procedural complexity. The trade-off is that a greater proportion of the rotational input energy is dissipated as frictional heat, resulting in lower overall energy efficiency—a compromise that is clinically acceptable given the very low actuation rates (0.5-1.0 mm/day) and the relatively small magnitude of input torque required (˜3 mN·m). Furthermore, this frictional resistance is beneficial because it acts as a passive damper, protecting the callus from abrupt unloading. It also enables a slip-clutch to be tuned just above the self-locking torque, thereby preventing over-distraction.

Geartrain Architecture and Kinematics: The geartrain drive consists of a primary mesh (worm stage) and a final mesh (spur stage). The primary mesh consists of a single-start worm driving a 26-tooth wheel (module m=0.2 mm). So, when z_{wheel} is the Number of teeth on the worm wheel and z_{worm} is the Number of starts (threads) on the worm—A single-start worm acts like a single-tooth helical gear wrapped around a cylinder. The primary gear ratio is

$${G}_{1} = \frac{{z}_{wheel}}{{z}_{worm}} = 26 \text{:1} ,$$

meaning that the worm must rotate 26 times to make the wheel rotate once. The secondary mesh consists of an 8-tooth pinion driving a 14-tooth gear (module m=0.5 mm). So, the secondary gear ratio is

$${G}_{2} = \frac{{z}_{wheel}}{{z}_{worm}} = 1.75 \text{:1} ,$$

meaning that the final spur gear rotates once for every 1.75 rotations of the pinion. Therefore, the total transmission ratio is

$$G = {G}_{1} {G}_{2} = 26 \times 1.75 = 45.5 : 1$$

Torque Flow and Efficiencies: Let T₀ be the input torque, T₁ the torque at the worm wheel and pinion shaft, and Tₒᵤₜ the output torque at the final spur gear. So, the input torque is:

$${T}_{0} = \frac{{T}_{out}}{G} ⟹ {T}_{0} = \frac{0.065}{45.5} = 1.43 \times {10}^{- 3} N \cdot m \approx 1.43 m \cdot Nm .$$

When we add the mesh efficiencies to the equation, as η₁˜0.50 for a small single-start worm, and η₂˜0.95 for the final spur gear, the input torque is:

$${T}_{0} = \frac{{T}_{out}}{{η}_{1} {η}_{2} G} ⟹ {T}_{0} \approx \frac{0.065}{0.5 \times 0.95 \times 45.5} \approx 3 \times {10}^{- 3} N \cdot m = 3 mN \cdot m .$$

The efficiency penalty will increase the input torque to approximately three mN·m, but it doesn't increase the frictional force since the frictional force is orthogonal to the tooth force.

For each mesh, the transmitted tangential tooth force can be calculated directly from torque and pitch diameter

$$( {d}_{p} ) : {W}_{t} = \frac{2 T}{{d}_{p}} .$$

For the primary mesh, when

$${T}_{1} = \frac{{T}_{out}}{{η}_{2} {G}_{2}} ⟹ {T}_{1} = \frac{0.065}{0.95 \times 1.75} = \frac{0.065}{1.6625} \approx 39.1 \times {10}^{- 3} N \cdot m = 39.1 mN \cdot m , and$$

$${d}_{p} = 5.2 mm , {W}_{t} = \frac{2 ( 0.03909 )}{0.0052} \approx 15.04 N .$$

For the spur gear, when Tₒᵤₜ=0.065N·m, and

$${d}_{p} = 4 mm , {W}_{t} = \frac{2 {T}_{out}}{{d}_{p}} = \frac{2 ( 0.065 )}{0.004} \approx 32.5 N .$$

Gear-Tooth Stress Analysis: To verify that the miniature gears tolerate the tangential tooth loads, we use the classical Lewis bending model. For a given mesh, the allowable tangential force (W_{t,allow}) carried at the pitch line (per tooth) is

$${W}_{t , allow} = \frac{SFY}{DP} ,$$

where S is allowable bending stress of the gear material (S=225 MPα for a conservative brass/bronze miniature gear assumption); F is face width (primary: F=2 mm; final: F=4 mm); Y is Lewis form factor (dependent on tooth count and pressure angle; Y=0.20 used here for a conservative small-tooth-count estimate); DP is diametral pitch. For primary mesh (worm wheel): module m=0.2 ⇒mm=DP=1/0.0002=5000 m⁻¹. For final mesh (spur gear): module m=0.5 ⇒mm=DP=1/0.0005=2000 m⁻¹.

Therefore, the allowable forces per the Lewis formula are:

For primary mesh (worm wheel):

$${W}_{t , allow} = \frac{( 225 \times {10}^{6} ) ( 0.002 ) ( 0.2 )}{5000} = 18. N$$

For final mesh (spur gear):

$${W}_{t , allow} = \frac{( 225 \times {10}^{6} ) ( 0.004 ) ( 0.2 )}{2000} = 90. N$$

Lewis bending stress can be computed as

$${σ}_{b} = \frac{{W}_{t} D P}{FY} ⇒ S F = \frac{S}{{σ}_{b}} = \frac{{W}_{t , allow}}{{W}_{t}}$$

For primary mesh (worm wheel):

$${σ}_{b} = \frac{15.04 \times 5000}{0.002 \times 0.20} = 188 MPa ⟹ SF = \frac{225}{188} \approx 1.2$$

For final mesh (spur gear):

$${σ}_{b} = \frac{32.5 \times 2000}{0.004 \times 0.20} = 81.25 MPa ⟹ SF = \frac{225}{81.25} \approx 2.77$$

Under the target torque of 0.065 N·m at the nut, the primary worm-wheel teeth experience a tangential load of ˜15.04N, yielding a Lewis safety factor of ˜1.2, whereas the final spur mesh carries ˜32.5N per tooth, providing a safety factor of ˜2.77. Although the primary mesh operates close to its allowable limit, the allowable limit itself is based on a conservative selection that provides a substantial margin before material yielding or fatigue occurs. In addition, the MAC system is subjected to a very low duty cycle (˜2500 worm revolutions over an entire distraction course) and is torque-capped by the slip-clutch, further reducing the risk of overload. For these reasons, a safety factor near unity on the worm wheel is acceptable for this clinical application. If an additional margin were desired, it could be achieved by modestly increasing the worm-wheel face width, selecting a higher Lewis form factor, or slightly lowering the maximum distraction torque—all simple design adjustments that would raise the safety factor without altering the footprint of the device.

Output Rotation⇒Linear Distraction: When the screw lead (l) is 0.6 mm and the total gear reduction (G) is 45.5:1 (45.5 input revs=one output rev), linear advance per input revolution is:

$$Δ {x}_{per rev} = \frac{l}{G} = \frac{0.6 mm}{45.5} = 13.2 µm .$$

Therefore, the total number of revolutions needed for 1 mm distraction is:

$${N}_{rev} = \frac{1. mm}{Δ {x}_{per rev}} = \frac{1.0}{0.01319} \approx 76 rev .$$

The subdermal geartrain requires 76 external magnet revolutions per one mm of distraction. While mechanically precise, these are far too many turns for practical daily use. To address this issue, the external handheld driver integrates an 8:1 rotation multiplier gearbox. This means that for every one revolution we perform, the driver magnet spins eight revolutions. The result is

$$\frac{76 rev / mm}{8} \approx 9.4 \sim 10 rev / mm .$$

This ration strikes a balance between precision and usability, similar to turning a pepper grinder.

Slip Clutch: The introduction of a slip-clutch mechanism into magnet-actuated cranial distraction (MAC) systems represents an advancement in ensuring mechanical reliability. In the MAC, externally applied torque is transmitted to generate an axial distraction force across the osteotomy. This force must remain within a narrow osteogenic window to promote robust intramembranous ossification while minimizing premature consolidation or fibrous non-union.

The slip clutch serves as a mechanical “circuit breaker,” designed to disengage torque transmission once a preset threshold is reached. By defining a fixed ceiling of transmitted torque, it enforces a maximum distraction force that is independent of the external magnetic field strength, soft-tissue standoff (scalp thickness), or inadvertent overactivation by caregivers. This is particularly crucial in pediatric craniofacial distraction, where even modest over-distraction can lead to dural tension, sinus kinking, or premature synostosis, while under-distraction risks callus collapse and treatment failure.

Mechanically, the slip clutch is implemented as a detent—or friction-based torque limiter integrated coaxially within the MAC driver. Under standard operation, the input torque (Tᵢₙ) from the driver is transmitted through the geartrain to the driving screw. Once the transmitted torque exceeds the calibrated limit Tₛₗᵢₚ, this “slipping” decouples further torque transmission and prevents additional axial load buildup. The governing equation for the torque limit can be expressed as

$${T}_{s l i p} = {F}_{max} ( {K}_{t h} + {K}_{c} ) ⟹ {F}_{max} = \frac{{T}_{slip}}{{K}_{t h} + {K}_{c}} ,$$

where Fₘₐₓ is the desired maximum axial distraction force, Kₜₕ is the thread torque constant (the amount of thread torque required per each newton of axial load for a given coefficient of friction), and K_{c} is the collar torque constant (the amount of collar torque per each newton of axial load necessary to overcome collar or thrust bearing friction for a given collar coefficient of friction and effective diameter). So, when the mean thread diameter (dₘ) is 3.25 mm, screw lead (l) is 0.6 mm, and coefficient of thread friction (f) is 0.35, the thread torque constant is given by:

$${K}_{t h} ( f ) = \frac{{d}_{m}}{2} \frac{l + π f {d}_{m}}{π {d}_{m} - f l} ⟹ {K}_{t h} ( 0.35 ) = \frac{0. 325}{2} \frac{0.0006 + π ( 0.35 ) ( 0.00325 )}{π ( 0.00325 ) - 0.35 ( 0.0006 )} = 6.78 \times 1 {0}^{- 4} N \cdot m / N$$

Therefore, every newton of axial load requires approximately 0.000678 N·m of thread torque.

Meanwhile, when the effective friction diameter (D_{c}) is 3.98 mm and the coefficient of friction (μ_{c}) is 0.10, the collar torque constant is expressed as:

$${K}_{c} ( {μ}_{c} ) = \frac{{μ}_{c} {D}_{c}}{2} ⟹ {K}_{c} ( 0.10 ) = \frac{0.10 ( 0.00398 )}{2} = 1.99 \times 1 {0}^{- 4} N \cdot m / N$$

Therefore, every newton of axial load requires approximately 0.000199 N·m of collar torque.

Torque-Limiting Safety Mechanism (Force Capping at 100N): The mechanical slip is calibrated to disengage at a precisely defined torque threshold (Tₛₗᵢₚ), thereby capping the maximum axial distraction force (Fₘₐₓ) that can be transmitted to the osteotomy. Therefore, Tₛₗᵢₚ must be computed under the lowest expected thread coefficient of friction (0.20). This “best-case” scenario yields the highest axial force per unit torque.

$${T}_{s l i p} = {F}_{max} ( {K}_{t h} + {K}_{c} ) = 100 ( 4.2549 \times 1 {0}^{- 4} + 1.99 \times 1 {0}^{- 4} ) = 0.062449 N \cdot m ( 62.45 mN \cdot m )$$

This corresponds to a maximum axial force of 71.18N and 59.58N at f=0.35, and 0.45, respectively.

$$F = \frac{0.062449}{6.7838 \times 1 {0}^{- 4} + 1.99 \times 1 {0}^{- 4}} \approx 71.18 N$$

$$F = \frac{0.062449}{8.4919 \times 1 {0}^{- 4} + 1.99 \times 1 {0}^{- 4}} \approx 59.58 N$$

This approach ensures that even under the most efficient (lowest-friction) conditions, such as early postoperative days with minimal soft-tissue impedance, the distraction force does not exceed the safe osteogenic window. The progressive increase in friction that accompanies callus maturation naturally derates the transmitted axial force. Calibrating Tₛₗᵢₚ based on the lowest plausible f and μ_{c} values, satisfies the principle of “safety-first design.” and ensures that no patient can be subjected to forces exceeding the predetermined maximum force limits.

Guarantee the Minimum Force at 74N: In the “guarantee-minimum” approach, Tₛₗᵢₚ is calibrated under the highest expected thread coefficient of friction (0.45) which is the least efficient (highest coefficient of friction) during clinical use.

$${T}_{s l i p} = {F}_{min} ( {K}_{t h} + {K}_{c} ) = 74 ( 8.4919 \times 1 {0}^{- 4} + 1.99 \times 1 {0}^{- 4} ) = 0.07756 N \cdot m ( 77.57 mN \cdot m )$$

This corresponds to a maximum axial force of 88.40N and 124.20N at f=0.35, and 0.20, respectively.

$$F = \frac{0.07756}{6.7838 \times 1 {0}^{- 4} + 1.99 \times 1 {0}^{- 4}} \approx 88.4 N$$

$$F = \frac{0.07756}{4.2549 \times 1 {0}^{- 4} + 1.99 \times 1 {0}^{- 4}} \approx 124.2 N$$

At this setting, the device reliably achieves at least Fₘᵢₙ=74N under the highest friction conditions. However, the distraction force rises significantly under low-friction conditions, reaching 88.4N at f=0.35 and 124.2N at f=0.20. This approach guarantees consistent distraction across the full spectrum of clinical conditions. Still, it does so at the expense of the safety margin, increasing the risk of overshooting and delivering excessive axial force.

Magnetic Force Modeling: A permanent magnet is composed of a material that retains intrinsic magnetization (\vec{M}) even after exposure to an external magnetic field. As a result, it generates a static magnetic field (\vec{B}) capable of attracting or repelling other magnets. The configuration of this magnetic field is determined by the shape of the magnet and the relative positioning of its north and south poles

A magnet can be modeled as a magnetic dipole oriented in the same direction as the magnetization vector, generating a dipolar magnetic field. This field is influenced by the permeability of free space (μ0) and the position vector (\vec{r}), which defines the location of the magnetic field relative to the magnetic dipole. According to the following equation, the strength of the magnetic field decreases as the distance from the magnet increases.

$$\vec{B} ( \vec{r} ) = \frac{μ 0}{4 π} ( \frac{3 r ( \vec{m} \cdot \vec{r} )}{r 5} - \frac{\vec{m}}{r 3} )$$

Torque is directly dependent on the distance between the point of force application and the axis of rotation. A greater distance results in higher torque for the same applied force, following the relationship (\vec{T}=r×F), where \vec{T} represents torque, r is the perpendicular distance from the axis of rotation, and F is the applied force. In the case of interaction between two magnets, the torque (\vec{T}) exerted by the external magnet (G1) on the internal magnet (G2) is given by \vec{T}=\vec{m}2×\vec{B}1 where \vec{m}2 is the magnetic Moment of the internal magnet, and \vec{B}1 denotes the magnetic field generated by the external magnet (G1) at the position of the internal magnet (G2).

In this device, we use two cylindrical magnets; each is diametrically magnetized and aligned along their principal axis. The external magnet must be precisely aligned with the internal magnet to activate the distraction process successfully. Thus, any deviation in alignment reduces the applied torque, following a cosine function of the misalignment angle. Specifically, at an angular deviation of 15°, the torque decreases by approximately 4%, whereas at 30°, it diminishes by nearly 15%. This highlights the importance of maintaining precise alignment to ensure optimal force transmission during distraction osteogenesis.

The torque exerted along this axis depends on several key parameters: (1) the cube of the distance between the magnets (r3); (2) the volume of both magnets (V1 & V2); (3) the magnetization of each magnet (\vec{M}1 &\vec{M}2); (4) the angle (α) between the magnetization vectors (\vec{M}1 &\vec{M}2). The maximum torque is generated when α=90°, while the torque is zero when the magnetization vectors are perfectly aligned (α=0° or) 180°.

The change in the magnetic field generates a torque (\vec{T}) on the internal magnet, which is directly transmitted to the driving rod without loss. This torque drives the rod's movement, resulting in the controlled divergence of the two baseplates affixed to the cranial bones, thereby facilitating distraction osteogenesis.

The protocol involved measuring the magnetic field produced by each magnet at a specified distance (r) using a longitudinal probe connected to a gaussmeter (GM08, Hirst Magnetics). The magnetization (\vec{M}) was then determined using the following formula: \vec{M}=2πr3\vec{B}/μ0 where (\vec{B}) represents the measured magnetic field, (r3) is the distance at which the measurement was taken, and (μ0) is the permeability of free space. This calculation allowed for an accurate estimation of the magnetization properties of the neodymium magnets used in the system.

To evaluate how the magnetic interaction influences the applied torque over varying separation distances, we calculated the torque (\vec{T}) applied to the internal magnet at a given distance between the magnets (r3) using the following equation:

$$\vec{T} \cdot \vec{u} z = \frac{\vec{M} 1 V 1 \cdot \vec{M} 2 V 2 μ 0}{2 π r 3} sin α$$

Rotary Magnetic Coupling: Inherent Force Self-limiting Behavior—A rotary magnetic coupling is inherently force self-limiting because torque transmission occurs purely through magnetic shear stress, rather than rigid mechanical contact. Once the transmitted torque (Tᵢₙₚᵤₜ) exceeds the maximum coupling torque (Tₘₐₓ), the system naturally decouples—the external and internal magnets slip relative to one another without further increase in transmitted torque.

In a typical coaxial magnet coupling, the magnets are arranged with opposite polarities so that the input torque arises from the tangential component of magnetic attraction between the corresponding poles. At low loads, both magnets remain synchronously locked by magnetic forces, and torque is transmitted smoothly as long as Tᵢₙₚᵤₜ<Tₘₐₓ. Once Tᵢₙₚᵤₜ exceeds Tₘₐₓ, the magnetic field lines can no longer maintain the alignment, and the poles slip. Therefore, there is an inherent built-in torque ceiling in magnet coupling when Tᵢₙₚᵤₜ=Tₘₐₓ, providing a natural overload protection.

Torsional Stiffness: Torsional stiffness (k_{θ}) is the proportionality constant between the input torque and slip angle (θ). In a magnet coupling, the external and internal magnets are held together only by magnetic attraction across an air gap. When we rotate the external magnet relative to the internal magnet, they become slightly misaligned. The resultant magnetic field then generates a restoring torque that increases almost linearly with the slip angle: Tᵢₙₚᵤₜ=k_{θ}θ, just like a torsional spring. So, torsional stiffness measures how strong and stiff a magnet coupling is.

Therefore, the magnitude of torsional stiffness has a direct impact on whether a magnetic coupling maintains its torque/self-limiting characteristic. So, in the case of high k_{θ}, the magnet coupling behaves almost like a rigid shaft, suppressing the elastic shear response and therefore eliminating the characteristic gradual torque-limiting behavior of magnet coupling—slip then occurs abruptly once Tₘₐₓ is exceeded. On the other hand, in the case of low k_{θ}, the magnet coupling is weak as the internal magnet lags behind the external magnet, which in turn leads to a gradual buildup of the transmitted torque, and when Tᵢₙₚᵤₜ<Tₘₐₓ, slip occurs gently and smoothly.

The design of the illustrative system 10 describe herein offers deterministic kinematics, bidirectional capability, predictable force generation, and built-in overload protection with haptic feedback. Designs in line with the system 10, therefore, represents a robust and clinically reliable solution for distraction systems, aligning with the overarching requirement of distraction osteogenesis: precise, incremental, and verifiable distraction or contraction of adjacent bone segments.

Distraction osteogenesis, simple terms, is a way to make a longer bone from a shorter bone by enabling bone formation between distracted bony segments. This distraction of adjacent bones is achieved through controlled traction to separate the target bony structures.

An exemplary distraction osteogenesis treatment incorporates cranial vault distraction devices to address craniofacial anomalies. Current cranial vault distraction devices require external activation ports protruding through the skin. The external activation ports are a significant source of biomechanical error by driving suboptimal placement of cranial distraction devices. Specifically, the required access to the activation port can substantially influence device orientation, thereby affecting the force vectors applied to the osteotomies. The external activation ports in current devices can also be associated with complications. Infection and wound-related complications are most common, followed by CSF leak and other device-related complications. Premature device removal and return to the operating room can be regularly required.

Craniosynostosis and other congenital craniofacial anomalies are examples of conditions that can benefit from distraction osteogenesis. Craniosynostosis is defined as the premature fusion of one or more cranial sutures. It affects 1 in 2,500 live births. Congenital craniofacial anomalies, such as midface hypoplasia, affect 1 in 5,600 live births. Syndromic craniosynostosis constitutes 15% of all cases and is often associated with systemic and extracranial abnormalities, depending on the syndrome involved. The surgical management of syndromic craniosynostosis include intracranial volume expansion, high intracranial pressure (ICP) reduction, orbital protection, and restoration of normal craniofacial anatomy. In some cases, distraction osteogenesis can be part of the surgical management for these conditions. It is contemplated that principles and design elements of the system 10 of the present disclosure can be applied to various different medical distraction applications.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

What is claimed is:

1. A medical distraction system, the system comprising
a distractor adapted to be fixed to bone segments and placed in its entirety under the skin of a patient, the distractor configured to displace one bone segment relative to another, the distractor including:
a fixed base configured to be fixed to a first bone segment,
a mobile base configured to be fixed to a second bone segment and having a rod that extends to the fixed base along a rod axis, and
a gear train configured to drive motion of the mobile base relative to the fixed base via interaction with the rod, the gear train configured to drive motion of the mobile base away from the fixed base via interaction with the rod, and
a driver adapted to remain in its entirety external to the patient and configured to drive motion of the distractor via magnetic coupling with the distractor.

2. The system of claim 1, wherein the gear train of the distractor includes an input magnet mounted for rotation about an input axis that is different from the rod axis.

3. The system of claim 2, wherein the gear train includes a worm gear fixed to the input magnet for rotation therewith.

4. The system of claim 3, wherein the gear train includes a drive nut having external gear teeth and internal threads engaged with the rod.

5. The system of claim 4, wherein the rod is formed to include threads engaged with the internal threads of the drive nut.

6. The system of claim 4, wherein the gear train includes a transfer gear assembly including a worm wheel engaged with the worm gear and a transfer gear having gear teeth engaged with the external gear teeth of the drive nut.

7. The system of claim 1, wherein the fixed base includes a base plate with holes therethrough sized to receive fasteners and a rod guide defining a passage along the rod axis sized to receive the rod therethrough.

8. The system of claim 7, wherein the rod guide includes a first frame having a first aperture defining a portion of the passage and a second frame having a second aperture defining another portion of the passage, the second aperture being spaced apart from the first aperture along the passage.

9. The system of claim 8, wherein the gear train is arranged to engage the rod along the rod axis between the first aperture and the second aperture.

10. The system of claim 9, wherein the gear train is fixed to the fixed base.

11. The system of claim 10, wherein the driver includes a housing, a knob coupled to the housing for rotation about a driver axis, and a drive magnet coupled to the knob for rotation about the driver axis and configured to magnetically couple to the input magnet of the distractor while the distractor is under the skin of the patient and the driver remains external to the skin of the patient.

12. The system of claim 11, wherein the driver includes a transmission coupled between the knob and the driver magnet, the transmission having a gearbox configured to convert a single rotation of the knob into more than one rotation of the drive magnet.

13. The system of claim 12, wherein the transmission includes a slip clutch configured to selectively decouple the drive magnet from the knob when torque developed between the drive magnet and the knob exceed a preselected threshold.

14. The system of claim 11, wherein the driver includes a transmission coupled between the knob and the driver magnet, the transmission having a slip clutch configured to selectively decouple the drive magnet from the knob when torque developed between the drive magnet and the knob exceed a preselected threshold.

15. The system of claim 11, wherein the driver includes a feedback unit configured to provide auditory and/or haptic feedback indicative of the amount of knob rotation relative to the housing so that a user can monitor expected rotation of the drive magnet along with the associated motion of the distractor during operation of the system.

16. The system of claim 15, wherein the feedback unit includes (i) a detent fixed to one of the knob or the housing and (ii) an elastic protrusion fixed to the other of the knob or the housing, the elastic protrusion arranged to be contacted by the detent upon rotation of the knob relative to the housing.

17. The system of claim 16, wherein the elastic protrusion is provided by a spring-loaded ball.

18. The system of claim 11, wherein the driver includes a magnet positioner unit configured to selectively change the distance from the driver magnet to an end of the housing, and wherein the magnet positioner unit is made up of a cap coupled to the housing body via cam slots shaped to move the cap along the driver axis in response to rotation of the cap about the driver axis.

19. A distractor adapted to be fixed to bone segments and placed in its entirety under the skin of a patient, the distractor configured to displace one bone segment relative to another, the distractor comprising
a fixed base configured to be fixed to a first bone segment,
a mobile base configured to be fixed to a second bone segment and having a rod that extends to the fixed base along a rod axis, and
a gear train configured to drive motion of the mobile base relative to the fixed base via interaction with the rod, the gear train having an input magnet configured to drive motion of the mobile base away from the fixed base via interaction with the rod, wherein the input magnet is mounted for rotation about an input axis that is different from the input axis.

20. The distractor of claim 19, wherein the gear train includes a worm gear fixed to the input magnet for rotation therewith and a drive nut having external gear teeth and internal threads engaged with external threads formed on the rod.
